(19) United States
(12) Patent Application Publication
(10) Pub. No.: US 2023/0233744 A1
(43) Pub. Date: Jul. 27, 2023

(54) BREAST MILK FILTRATION DEVICE AND METHOD

(71) Applicant: Kaehler Science for Women, LLC, Maple Grove, MN (US)

(72) Inventor: Laura Clendenin, Maple Grove, MN (US)

(21) Appl. No.: 18/158,513

(22) Filed: Jan. 24, 2023

Related U.S. Application Data
(60) Provisional application No. 63/302,318, filed on Jan. 24, 2022.

(51) Int. Cl. A61M 1/06; A61K 31/53

(52) U.S. Cl. CPC A61M 1/062; A61K 31/53

(57) ABSTRACT

A system and method configured to separate PFAS from expressed breast milk, thereby producing breast milk which is essentially free (or at least contains a lesser amount of) per- and polyfluorinated substances, the system and method including filtration device including an adapter configured to be at least one of positioned in-line in a breast pump or between breast milk containers, and including a replaceable cyclodextrin based filter for filtration of PFAS from pumped breast milk.

### CROSS-REFERENCE TO RELATED APPLICATION

The present application claims priority to U.S. Provisional Application No. 63/302,318, filed Jan. 24, 2022, the entirety of which is incorporated herein by reference.

### TECHNICAL FIELD

The present disclosure relates generally to removal of perfluorochemicals (PFCs), and more particularly to a filtration device and method for removing per- and polyfluorinated substances (PFAS) from pumped breast milk.

### BACKGROUND

Per- and polyfluorinated substances (PFAS) (sometimes referred to as perfluorochemicals (PFCs)) are a group of man-made chemicals that includes PFOA, PFOS, GenX, and many other chemicals. PFAS have been manufactured and used in a variety of industries around the globe, including in the United States since the 1940s. CFAS are commonly used to make coatings and products that resist heat, oil, stains, grease, and water. Fluoropolymer coatings can be used in such varied products as clothing, furniture, adhesives, food packaging, heat-resistant non-stick cooking surfaces, and the insulation of electrical wire. Many chemicals in this group have been a concern because they do not break down in the environment, can move through soils and contaminate drinking water sources, and they build up (bioaccumulate) in fish and wildlife. PFAS have been found in rivers and lakes and in many types of animals on land and in the water.

There are a variety of ways that people can be exposed to PFAS. For example, people can be exposed to low levels of PFAS through food, which can become contaminated through contaminated soil and water used to grow the food, food packaging containing PFAS, and equipment that uses PFAS during food processing. In some instances, drinking water can be a source of exposure in communities where these chemicals have contaminated water supplies. People can also be exposed to PFAS chemicals if they are released during normal use, biodegradation, or disposal of consumer products that contain PFAS. People may also be exposed to PFAS used in commercially-treated products, such as carpets, leather and apparel, textiles, paper and packaging materials, and non-stick cookware.

Studies of laboratory animals given large amounts of PFAS have found that some PFAS may affect growth and development, reproduction, thyroid function, the immune system, and injure the liver. If humans, or animals, ingest PFAS (by eating or drinking food or water than contain PFAS), the PFAS are absorbed, and can accumulate in the body. PFAS stay in the human body for long periods of time. As a result, as people get exposed to PFAS from different sources over time, the level of PFAS in their bodies may increase to the point where they suffer from adverse health effects.

One potentially susceptible segment of the population to adverse effects from PFAS include the very young, particularly those less than one year old who may receive a significant portion of their nutrients through breast milk. Even where a mother (or other source of the breast milk) may take precautions to avoid PFAS consumption, previous exposure to PFAS may have caused a buildup over time, which can pass through to the breast milk. The present disclosure addresses this concern.

### SUMMARY OF THE DISCLOSURE

Embodiments of the present disclosure provide a system and method configured to separate PFAS from breast milk, thereby producing breast milk which is essentially free (or at least contains a lesser amount of) per- and polyfluorinated substances. In particular, embodiments of the present disclosure provide a filtration device including an adapter configured to be positioned in-line in a breast pump or between breast milk containers including a replaceable cyclodextrin based filter for removal of PFAS from pumped breast milk.

One embodiment of the present disclosure provides for a filtration device configured to be positioned between first and second collection units, the filtration device coupled to at least one of the first or second collection unit with either adapted to include a quantity of breast milk that may contain a quantity of PFAS, and including a filter reduces the quantity of PFAS contained within the breast milk when the breast milk is transferred from on collection unit to the other. In another embodiment the present disclosure provides for a method of filtering PFAS from breast milk including the steps of positioning a filtration device between first and second collection units, and transferring the breast mild from one collection unit to the other with the filter reducing the quantity of PFAS in the breast milk.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 depicts a cross-sectional view depicting a filtration device, in accordance with an embodiment of the disclosure.

FIG. 2 depicts a perspective view depicting a filter within a filtration device, including cyclodextrin, in accordance with an embodiment of the disclosure.

FIG. 3 depicts a perspective view of an alternative embodiment of a filtration device, in accordance with embodiments of the disclosure.

FIG. 4 depicts a cross-sectional view depicting an alternative embodiment of a filtration device, in accordance with embodiments of the disclosure.

FIG. 5 depicts a partial, cross-sectional view depicting the filtration device of FIG. 4, in accordance with embodiments of the disclosure.

FIG. 6 depicts a perspective view illustrating a filtration device including a cyclodextrin filter, in accordance with embodiments of the disclosure.

FIG. 7 depicts a perspective view of the filtration device of FIG. 6, in accordance with embodiments of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

Reference now will be made in detail to embodiments of the disclosure, one or more examples of which are illustrated in the drawings. Each example is provided by way of explanation in the disclosure and is not limited thereto. In fact, it will be apparent to those skilled in the art that various modifications and variations can be made in the present disclosure without departing from the scope of the disclosure. For instance, features illustrated or described as part of one embodiment can be used with another embodiment to yield a still further embodiment. Thus, it is intended that the present disclosure covers such modifications and variations as come within the scope of the appended claims and their equivalents. As used herein, the term “or” is generally intended to be inclusive (i.e., “A or B” is intended to mean “A or B or both”). The terms “first,” “second,” and “third” may be used interchangeably to distinguish one component from another and are not intended to signify location or importance of the individual components.

Throughout the specification, and in the claims, the term “connected” means a direct electrical, mechanical, or magnetic connection between the things that are connected, without any intermediary devices. The terms “coupled” or “integrated” mean either a direct electrical, mechanical, or magnetic connection between the things that are connected or an indirect connection through one or more passive or active intermediary devices. The term “circuit,” “module,” or “mechanism” may refer to one or more passive and/or active components that are arranged to cooperate with one another to provide a desired function.

The terms “substantially,” “close,” “approximately,” “near,” and “about” generally refer to being within +/−10% of a target value. Unless otherwise specified the use of the ordinal adjectives “first,” “second,” and “third,” etc., to describe a common object, merely indicate that different instances of like objects are being referred to and are not intended to imply that the objects so described must be in a given sequence, either temporally, spatially, in ranking, or in any other manner. It is to be understood that the terms so used are interchangeable under appropriate circumstances such that the embodiments of the disclosure described herein are, for example, capable of operation in other orientations than those illustrated or otherwise described herein.

The terms “left,” “right,” “front,” “back,” “top,” “bottom,” “over,” “under,” and the like in the description and in the claims, if any, are used for descriptive purposes and not necessarily for describing permanent relative positions.

As described herein, embodiment of the present disclosure provides systems and methods related to instruments or tools used to perform surgery on a patient and, more particularly, to surgical instruments with enhanced ultrasonic tissue dissection.

Breast pumps for use by nursing mothers are well known. They allow the nursing woman to express the breast milk as necessary or convenient, and further provide collection of the breast milk for later use. There are three general broad classifications of breast pumps: (1) hand pumps that generate suction manually, (2) battery operated pumps with small motors that generate suction from power supplied by batteries, and (3) electric pumps in which suction is created by various types of electric motors that run off a 120V alternating current supply. Some pumps can cross over these broad classifications. Various example breast pumps are disclosed in U.S. Pat. No. 8,070,715, the contents of which are incorporated by reference herein to the extent that they do not conflict with the contents of this disclosure.

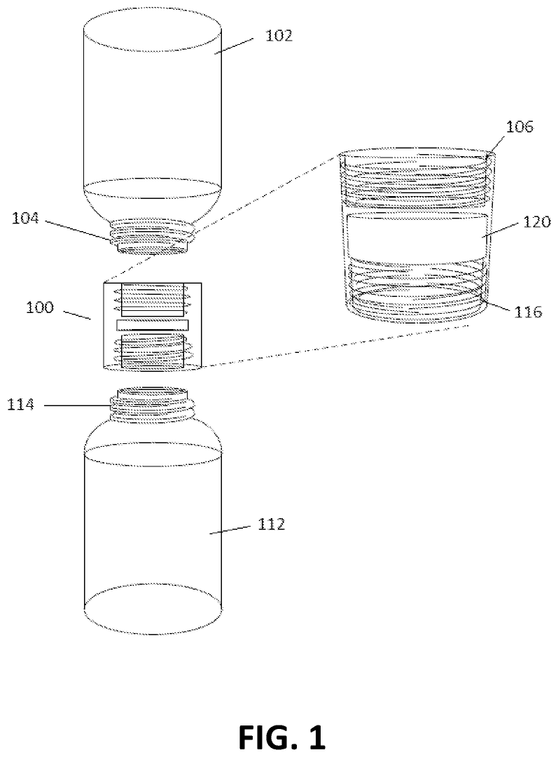

Embodiments of the present disclosure provide a system and method configured to filter or otherwise separate PFAS from expressed breast milk. In particular, with reference to FIGS. 1-3, in some embodiments, a filtration device 100 can be provided as an adapter configured to be positioned between a first collection bottle 102 and a second collection bottle 112. In some embodiments, the filtration device 100 can define a filter 120 in between a first internal threading 106 and a second internal threading 116 configured to threadably couple to an existing threaded portion 104 of the respective first collection bottle 102 and an existing threaded portion 116 of a second collection bottle 112; either of the first or second collection bottles 102 or 112 including a quantity of breast milk potentially containing a quantity of PFAS. In some embodiments, the first and second internal threading portions 106 and 116 may be substantially identical, such that either of the first or second internal threaded portions 106 and 116 may be coupled to either of the first or second collection bottles 102 or 112. In other embodiments, the first and second internal threaded portions 106 and 116 may be specific to a first, unfiltered breast milk container and a second, filtered breast milk container (e.g., from the first collection bottle 102 to the second collection bottle 112) as an aid in confirming that the breast milk has been filtered.

In embodiments, a first collection bottle 102 may contain an amount of unfiltered breast milk, wherein the first collection bottle 102 may be coupled to the filtration device 100. In embodiments, the filtration device 100 and first collection bottle 102 may be threadably coupled together by the first existing threading 104 and the first internal threading 106, forming a liquid seal. In embodiments, where there may not be threading (e.g., on either the first collection bottle 102 or the filtration device 100), the filtration device 100 may receive (e.g., slide onto) the first collection bottle 102, forming a liquid seal. In embodiments, the filtration device 100 and second collection bottle 112 may be threadably coupled together by the second existing threading 114 and the second internal threading 116, forming a liquid seal. In embodiments, where there may not be threading (e.g., on either the second collection bottle 112 or the filtration device 100), the filtration device 100 may receive the second collection bottle 112, forming a liquid seal.

In embodiments, once at least the first collection bottle 102 is in fluid communication with the filter device 100 (and the second collection bottle 112, if attached), the first collection bottle 102 may be tilted or inverted. Thus, the liquid within the first collection bottle 102 (e.g., the unfiltered breast milk) may flow from the first collection bottle 102, through the filter 120, and, thus, through the filtration device 100. In embodiments, the filtration device 100 may rely on gravity to process the unfiltered breast milk through filter 120. In other embodiments, the breast milk can be pressurized as an aid in causing the breast milk to flow through the filtration device 100. Other embodiments are also contemplated. In embodiments, the filtration device 100, particularly the filter 120, may filter out PFAS and other micropollutants that may be retained in the unfiltered breast milk or other liquid. In embodiments, the second collection bottle 116 may also be fluidly coupled to the filtration device 100 to receive the now filtered or becoming filtered breast milk, in real time, from the first collection bottle 102. In embodiments, not only are the PFAS and other micropollutants removed, the filtration device 100 may filter the breast milk without altering (or minimally altering) the nutritional profile of the breast milk. In embodiments, the filtration device 100 can reduce PFAS concentration in breast milk by up to 85%. In embodiments, the filtration device 100 can reduce PFAS concentration in breast milk by up to 90%. In embodiments, the filtration device 100 can reduce PFAS concentration in breast milk by greater than or equal to 90%. In embodiments, the filtration device 100 can reduce PFAS concentration in breast milk by greater than 99%. In embodiments, the reduction or removal of PFAS concentration may be performed while minimally altering the milks nutritional profile by 5% or less.

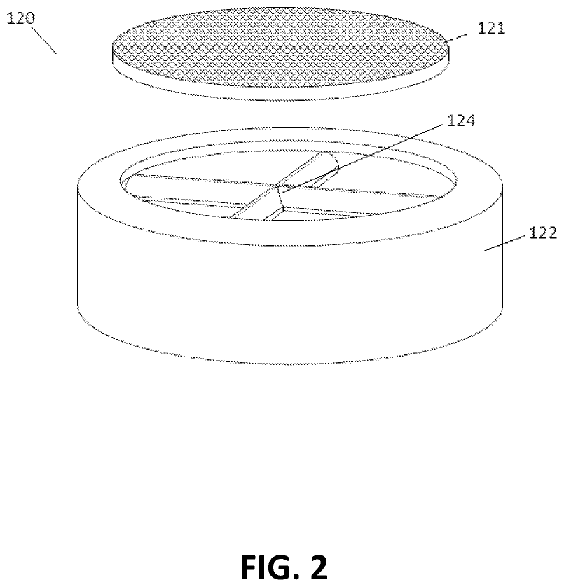

With additional reference to FIG. 2, FIG. 2 depicts a perspective view illustrating a filter within a filtration device, including cyclodextrin, in accordance with embodiments of the disclosure. In some embodiments, the filtration device 100 may include a filter 120. In embodiments, one or more surfaces 124 may be integrated and configured to support cyclodextrin 121 within the filter 120 of the filtration device 100. In some embodiments, the filtration device 100 can include the one or more surfaces 124 as a brace (e.g., in the form of a cross, etc.) configured to retain the cyclodextrin 121 in position relative to the filtration device 100 during operation. In embodiments, the one or more surfaces 124 may be integrated or otherwise attached to the inner surface of the wall 122 of the filtration device 100. In some embodiments, the filter 120 (e.g., the wall 122, one or more surfaces 124, etc.) can be constructed of a plastic or other suitable material. In embodiments, the filter 120 may be positioned in-between the first and second internal threading 106 and 116, wherein the filter 120 has a thickness that expands up to the leading edges of the first and/or the second internal threading 106 and/or 116. In embodiments, the filter 120 may have a thickness less than the length between the first internal threading 106 and the second internal threading 116. In embodiments, while the one or more surfaces 124 are depicted in a cross configuration, other configurations are contemplated. In some embodiments, the filter 120 can be configured for readable replacement between subsequent filtrations.

In some embodiments, the filter 120 and corresponding filters as illustrated herein can generally include cyclodextrin 121. Cyclodextrins are a family of cyclic oligosaccharides, consisting of a macrocyclic ring of glucose subunits joined by α-1,4 glycosidic bonds. Cyclodextrins are produced from starch by enzymatic conversion. Cyclodextrins are composed of 5 or more α-D-glucopyranoside units linked 1->4, as in amylose (a fragment of starch). The largest cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring. With a hydrophobic interior and hydrophilic exterior, cyclodextrins form complexes with hydrophobic compounds. Alpha-, beta-, and gamma-cyclodextrin are all generally recognized as safe by the U.S. FDA. In some embodiments, the cyclodextrin can be a powder form contained between two layers of retaining tissue or fabric. In some embodiments, the filter can generally be in the form of a patch, which can be selectively positioned within the filtration device 100. In embodiments, the filter 120 can generally be in the form of a disc like shape, which can be selectively positioned within the filtration device 100. In embodiments, the filter 120 can be incorporated with cyclodextrin in a compact or composed, single unit such that the filter 120 may be interchangeable and readily removed for cleaning or replaced.

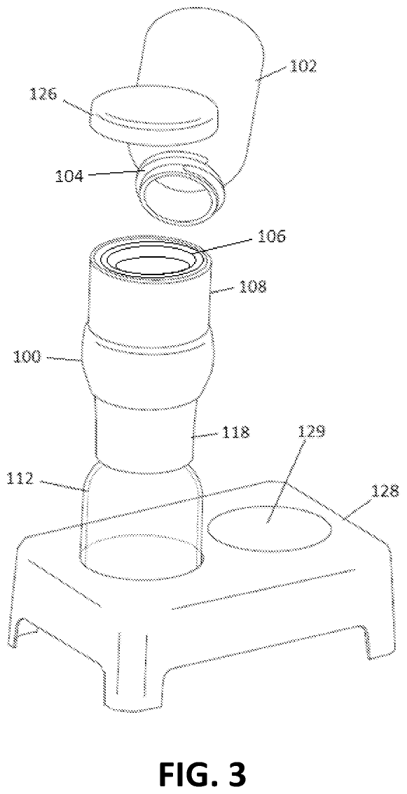

Referring to FIG. 3, in embodiments, similarly depicted in FIG. 1, the filtration device 100 may include additional or alternative configurations for breast milk filtration, in accordance with embodiments of the disclosure. In embodiments, extensions 108 and 118 may be included to receive collection bottles 102 and 112. In embodiments, extensions 108 and 118 may provide additional space within, configured to receive and hold a greater quantity of breast milk, while filtration progresses through the filtration device 100. In embodiments, the first internal threading 106 and second internal threading (not shown) may be configured to extend toward each respective edge of the attachments 108 and 118, resulting in empty space (i.e., greater capacity for reception and delivery of breast milk) between the collection bottles 102 and 112 and the filtration device 100. In embodiments, the first internal threading 106 and second internal threading (not shown) may be configured to threadably receive the first collection bottle 102, via first external threading 104 and the second collection bottle 112, via second external threading (not shown), which create liquid seals. In embodiments, optional lids 126 may be configured to seal both sides of attachments 108 and 118. The lids 126 configured to seal the filtration device 100 (i.e., keep out the external environment), thus keeping the filtration device 100 (and the filter 120) clean between uses. For example, after use, the filter 120 may be removed, cleaned and returned. In embodiments, the filter 120 may be replaced with a new filter and the lids 126 may be attached to keep the filter 120 clean/new until the next use of the filtration device 100. In embodiments, an optional stand 128 may be included to hold any collection bottles 102 or 112. While the stand 128 may be depicted with two openings 129 to receive collection bottles 102 or 112, alternative quantities are contemplated.

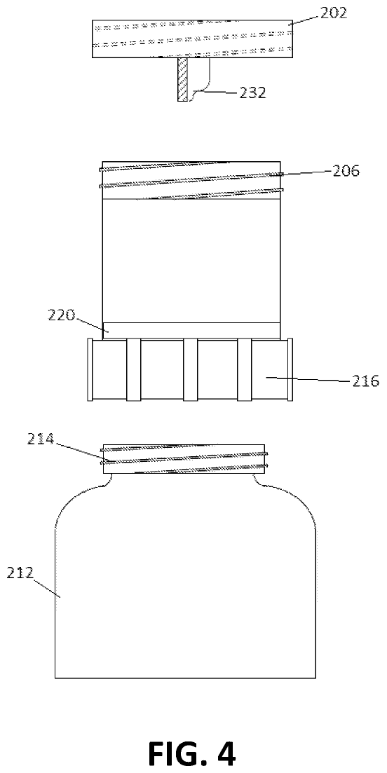

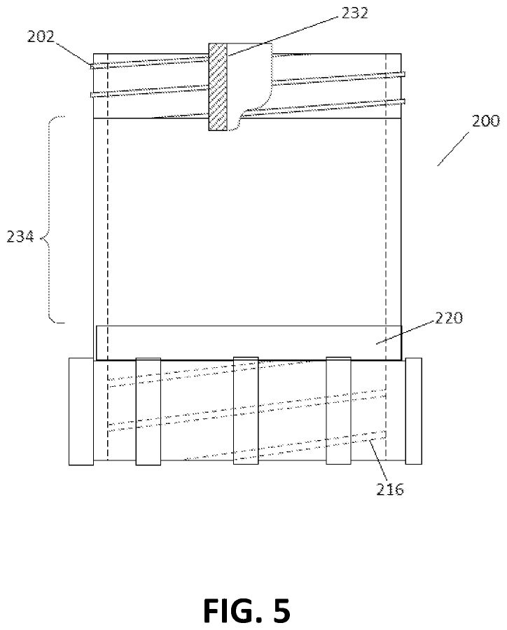

Referring to FIGS. 4-5, a filtration device may be configured to be positioned in-line with or within a breast pump, in accordance with an embodiment of the disclosure. Similar to the disclosure described in reference to FIGS. 1-3 and 6-7, the filtration device can include internal threading configured to threadably couple onto existing threaded portions of the respective breast pump, including a collection device or bottle.

FIG. 4 depicts an alternative embodiment of the filtration device, similarly depicted in FIGS. 1-3 for breast milk filtration, in accordance with embodiments of the disclosure. In embodiments, a filtration device 200 can be provided as an adapter configured to be positioned between a first collection device, for example a breast pump (not depicted), connectible by connector 202 and a collection bottle 212. In some embodiments, the filtration device 200 can define a filter 220 (further depicted in FIG. 5) positioned between a first threading 206 and a second threading 216 configured to be threadably coupled onto connector 202 and an existing threaded portion 216 of a collection bottle 212; the connector 202 configured to deliver unfiltered breast milk from a breast pump (not shown) through the filtration device 200 and received by collection bottle 212. In embodiments, the quantity of breast milk potentially containing a quantity of PFAS, which may be filtered out by filtration device 200. In some embodiments, the first and second threading portions 206 and 216 may be substantially identical, such that either of the first or second threaded portions 206 and 216 may be coupled to either of the connector 202 or the collection bottle 216. In other embodiments, the first and second threading portions 206 and 216 may be specific to a connector 202 and a breast milk container as an aid in confirming attachability (i.e., as a visual aid to identify which end of the filtration device connects to the connector 202/breast pump and which end connects to the collection bottle 212).

In embodiments, the connector 202 may distribute, from a breast pump, an amount of unfiltered breast milk, wherein the connector 202 may be coupled to the filtration device 200. In embodiments, the filtration device 200 and connector 202 may be threadably coupled together between the threading on the connector (not shown) and the first internal threading 206, forming a liquid seal. In embodiments, where there may not be threading (e.g., on either the connector 202 or the filtration device 200, the filtration device 200 may receive (e.g., slide onto) the connector 202, forming a liquid seal. In embodiments, the filtration device 200 and collection bottle 212 may be threadably coupled together between the second existing threading 214 and the second internal threading 216, forming a liquid seal. In embodiments, where there may not be threading (e.g., on either the collection bottle 212 or the filtration device 200, the filtration device 200 may receive the collection bottle 212, forming a liquid seal.

In embodiments, once at least the connector 202 is in fluid communication with the filtration device 200 (and the collection bottle 212, if attached) and the breast pump, the breast pump may be activated. In embodiments, the filtration device 200 may filter the unfiltered breast milk, in real-time (i.e., continuously, during extraction of breast milk from the breast pump). In embodiments, the filtration device 200, particularly the filter 220 (further depicted in FIG. 5), may filter out PFAS and other micropollutants that may be retained in the unfiltered breast milk. In embodiments, the collection bottle 212 may also be fluidly coupled to the filtration device 200 to receive the now filtered or becoming filtered breast milk, in real time, from the breast pump, which may be coupled to connector 202. In embodiments, not only are the PFAS and other micropollutants removed, the filtration device 200 may filter the breast milk without changing (or minimally changing) the nutritional profile of the breast milk. In embodiments, the filtration device 200 can reduce PFAS concentration in breast milk by up to 85%. In embodiments, the filtration device 200 can reduce PFAS concentration in breast milk by up to 90%. In embodiments, the filtration device 200 can reduce PFAS concentration in breast milk by greater than or equal to 90%. In embodiments, the filtration device 200 can reduce PFAS concentration in breast milk by greater than 99%. In embodiments, the reduction or removal of PFAS concentration may be performed while minimally altering the milks nutritional profile by 5% or less.

In some embodiments, the filtration device 200 can include a valve 232 configured to enable gas that may be trapped within the collection bottle 212 or filtration device 200 to escape as breast milk flows into the collection bottle 212 and displaces the gas. In embodiments, the filtration device 200 may filter the breast milk during operation of the breast pump without impeding pump-flow rate and without impeding (or minimally impeding) comfort to the user.

With additional reference to FIG. 5, FIG. 5 depicts a partial, cross-sectional view illustrating the filtration device of FIG. 4, in accordance with embodiments of the disclosure. In embodiments, threading portion 206 may be a threaded exterior to the filtration device 200, in which connector 202 may be coupled. Thus, providing a smooth receiving interior for the unfiltered breast milk. At the opposing end of the filtration device 200, similarly depicted, internal threading 216 may be integrated, which provides a smooth exterior at the opposing end of the filtration device 200. In embodiments, the filter 220 may be position at the end closest to the internal threading 216, such that the received breast milk may flow through an air gap 234. While the filter 220 may be illustrated in a particular location within the filtration device 200, other embodiments are also contemplated. The air gap 234 providing more efficient flow of unfiltered breast milk. In embodiments, the filtration device 200 may rely on gravity to process the unfiltered breast milk through filter 220. In other embodiments, the breast milk can be pressurized as an aid in causing the breast milk to flow through the filtration device 200. Thus, as unfiltered breast milk is received, a build up (or partial build up) of unfiltered breast milk is contained in the air gap 234 and may steadily flow through the filter 220 and into the collection bottle 212. Other embodiments are also contemplated. In embodiments, as breast milk is being filtered, any gas that may be trapped within the collection bottle 212 or the air gap 234 of the filtration device 200 may escape out through valve 232. In some embodiments, similar to FIGS. 1-2, the filter 220 and other filters as illustrated herein can generally include cyclodextrin, aiding in the removal of PFAS and other micropollutants that may be retained in the unfiltered breast milk or other liquid passing thought the filter 220, while maintaining the nutritional integrity of the breast milk.

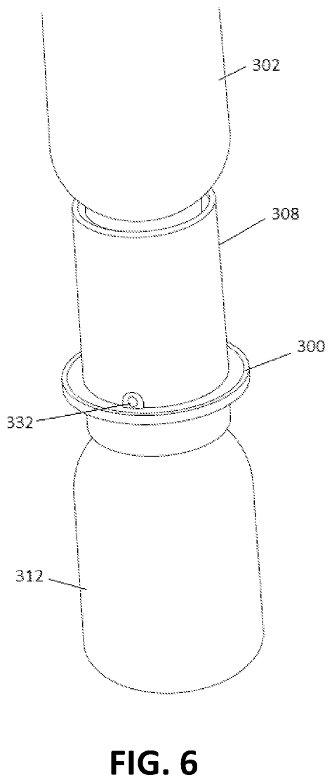

FIG. 6 depicts an alternative embodiment of the filtration device, similarly depicted in FIGS. 1-3 for breast milk filtration, in accordance with embodiments of the disclosure. In embodiments, a filtration device 300 can be provided as an adapter configured to be positioned between a first collection bottle 302 and a second collection bottle 312. In some embodiments, the filtration device 300 can be configured to be threadably coupled onto the respective first collection bottle 302 and a second collection bottle 312; either of the first or second collection bottles 302 or 312 including a quantity of breast milk potentially containing a quantity of PFAS.

In embodiments, a first collection bottle 302 may contain an amount of unfiltered breast milk, wherein the first collection bottle 302 may be coupled to the filtration device 300, via an extension 308, similarly depicted in FIG. 3. In embodiments, the filtration device 300 and first collection bottle 102 may be threadably coupled together, forming a liquid seal. In embodiments, an air gap, similar depicted in FIG. 5, may be incorporated within the extension 308 threadably connected between the first collection bottle 302 and the filtration device 300. In embodiments, where there may not be threading (e.g., on either the first collection bottle 102, the extension 308 or the filtration device 300), the filtration device 300 and extension 308 may receive (e.g., slide onto) the first collection bottle 302, forming a liquid seal. In embodiments, the filtration device 300 and second collection bottle 312 may be threadably coupled together, forming a liquid seal. In embodiments, where there may not be threading (e.g., on either the second collection bottle 312 or the filtration device 300), the filtration device 300 may receive the second collection bottle 312, forming a liquid seal.

In embodiments, once at least the first collection bottle 302 is in fluid communication with the filtration device 300, via the extension 308, (and the second collection bottle 312, if attached), the first filtration device 302 may be tilted or inverted. Thus, the breast liquid within the first collection bottle 302 (e.g., the unfiltered breast milk) may flow from the first collection bottle 302, through extension 308, potentially building up within, and through the filter 300. In embodiments, the filtration device 300 may rely on gravity to process the unfiltered breast milk through filter 320. In other embodiments, the breast milk can be pressurized as an aid in causing the breast milk to flow through the filtration device 300. Other embodiments are also contemplated. In embodiments, the filtration device 300, particularly the filter (not shown), may filter out PFAS and other micropollutants that may be retained in the unfiltered milk or other liquid. In embodiments, the second collection bottle 316 may also be fluidly coupled to the filtration device 300 to receive the now filtered or becoming filtered breast milk, in real time, from the first collection bottle 302. In embodiments, not only are the PFAS and other micropollutants removed, the filtration device 300 may filter the breast milk without changing (or minimally changing) the nutritional profile of the breast milk. In embodiments, the filtration device 300 can reduce PFAS concentration in breast milk by up to 85%. In embodiments, the filtration device 300 can reduce PFAS concentration in breast milk by up to 90%. In embodiments, the filtration device 300 can reduce PFAS concentration in breast milk by greater than or equal to 90%. In embodiments, the filtration device 300 can reduce PFAS concentration in breast milk by greater than 99%. In embodiments, the reduction or removal of PFAS concentration may be performed while minimally altering the milks nutritional profile by 5% or less. In some embodiments, the filtration device 300 can include a valve 332 configured to enable gas that may be trapped within the collection bottle 312 to escape as breast milk flows into the second collection bottle 312 and displaces the gas.

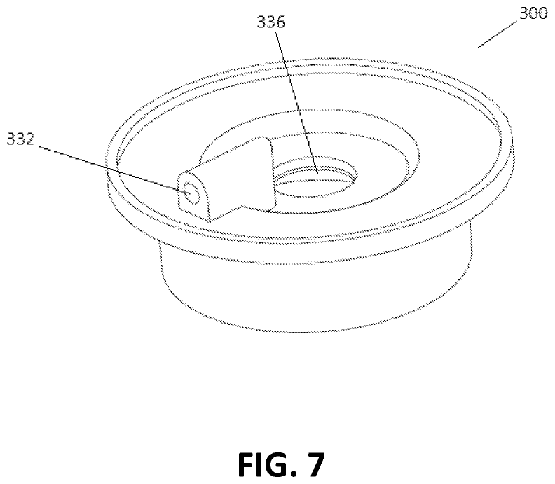

With additional reference to FIG. 7, FIG. 7 depicts a perspective view of the filtration device of FIG. 6, in accordance with embodiments of the disclosure. In embodiments, the filter, as similarly described herein, may be retained within the filtration device 300 as a sole, interchangeable, compact unit. In embodiments, the filtration unit 300 may be readably attachable and detachable to the first collection bottle 302, extension 308, and the second collection bottle 312, for example, by each of their threadable connectivity. For example, the filtration device 300 may be directly, threadably connected to the first filtration bottle 302 and the second collection bottle 312. In embodiments, where the first collection bottle retains at least a quantity of unfiltered breast milk, the system may be tilted or inverted (as depicted in FIG. 6), in which breast milk may flow directly through the filtration device 300, through opening 336, and into the second collection bottle 312. In some embodiments, the filtration device 300 can include a valve 332 configured to enable gas that may be trapped within the collection bottle 312 to escape as breast milk flows into the collection bottle 212 and displaces the gas. In some embodiments, similar to FIGS. 1-2, the filter and other filters as illustrated herein can generally include cyclodextrin, aiding in the removal of PFAS and other micropollutants that may be retained in the unfiltered breast milk or other liquid passing thought the filter, while maintaining the nutritional integrity of the breast milk.

### Example Study Analysis

Rigor of Prior Research

Previous studies on PFAS contamination in breast milk was important, foundational work linking breastfeeding to infant exposure to PFAS. However, these studies offer limited recommendations for intervention and lag behind the current regulatory reality. Several studies have linked increased PFAS concentrations in the serum of reproductive age and pregnant women to dietary factors like consumption of seafood or tap (versus bottled) water. This work has led to recommendations for lifestyle changes to limit further PFAS exposure like filtering drinking water, checking local fish advisories before consuming locally sourced fish or seafood, and avoiding household products known to contain PFAS. However, these recommendations only address future, additional exposure to PFAS in the body of a breastfeeding mother and do not address prior accumulation. These studies also lag behind the current regulatory climate as they do not include newer PFAS compounds without manufacturing restrictions, like GenX. Our study investigated the efficacy of an intervention strategy to address prior accumulation in the body of a breastfeeding mother and includes the newer, less restricted GenX.

Innovation

Despite the well-documented contamination of breast milk with PFAS, there has been no current tests or products for mothers to address this issue. Trusted authorities, like the CDC, acknowledge that PFAS contamination in breast milk is a problem but state that the benefits of breastfeeding outweigh the problems associated with PFAS exposure. Even state-of-the-art breast milk testing, like that provided by the Lactation Lab, does not address PFAS contamination in breast milk. Unlike many nutritional deficiencies, there is nothing a nursing mother can do to lower the PFAS concentration resulting from accumulation throughout her life. Current filtration technologies for removing PFAS from water, such as carbon, ion exchange, reverse osmosis, and cyclodextrin, have never been applied to breast milk. These technologies have been proven effective in drinking water matrices and are recommended to nursing mothers to mitigate additional PFAS exposure. In embodiments, the filtration device as illustrated herein aids in PFAS removal from breast milk and addresses PFAS already accumulated in the bodies of nursing mothers.

The filter embodiments, as described herein, may be composed of cyclodextrin, a non-toxic, naturally occurring sugar that has been shown to remove 99% of PFOS from water. The use of technology with a non-toxic, natural media prevents the introduction of leachates that may be toxic or synthetic. Utilizing cyclodextrin as the filtration media, versus the drinking water industry's common technologies of carbon, ion exchange, and reverse osmosis, also allows an increased specificity for PFAS compounds. The addition of amine groups to the cyclodextrin molecule greatly increases the specificity and binding affinity for PFAS, including short- and long-chain compounds. Whereas other technologies rely on size and charge filtration, which would likely impact many other nutrients and microbial components of the complex matrix of breast milk, cyclodextrin's unique molecular structure and specificity for PFAS minimizes the incidental filtration of beneficial components of the breast milk matrix. This functionality makes cyclodextrin an ideal media, as it will remove PFAS without interfering with the major nutritional benefits of breast milk. Analysis can be found in Table 1 below.

TABLE 1
Competitive Landscape for PFAS Filters for Breast milk and Formula
Review of available technologies utilized in drinking water PFAS remediation.
EPA health advisory (HA) level of 70 ppt for PFOA, PFOS.

| | | | | Breast | | Cost |
|---|---|---|---|---|---|---|
| Product | Removal | Removal | Filtration | Milk | Targets PFAS | per |
| (Manufacturer) | Mechanism | Efficiency | location | Compatible | Accumulation | use |
| Kaehler Science for | Cyclodextrin | 85% (Phase I) | Breast pump | Yes | Yes | $ |
| Women | | 99.7% (Phase II) | or bottle | | | |
| eSpring Water | Carbon and | EPA HA level | Point of use | No | No | $ $ |
| Treatment (Amway) | UV light | [70 ppt] | | | | |
| The Clean Water | Carbon, ion | EPA HA level | Point of use | No | No | $ |
| Filter (A. O. Smith) | exchange | [70 ppt] | | | | |
| PFA694E (Purolite) | Ion | EPA HA level | Point of use | No | No | $ $ $ |
| | exchange | [70 ppt] | | | | |
| | resin | | | | | |
| E-series (Suez) | Reverse | EPA HA level | Point | No | No | $ $ |
| | osmosis | [70 ppt] | of | | | |
| | | | entry | | | |

Preliminary Data:

Analyses for both the measure PFAS removal efficiency with the filtration device, and the monitoring of the nutritional profile of breast milk before and after filter use, from one breast milk donor is provided in Tables 2 and 3 below. PFAS quantification analyses were run in triplicate pre- and post-filtration (Table 2). PFOS and GenX were below the limit of detection in this breast milk sample. The PFAS removal efficiency of cyclodextrin has also been measured in water at comparable concentrations (Table 2). Breast milk nutritional analyses were conducted on a single sample pre- and post-filtration (Table 3).

TABLE 2
Preliminary Results - Effective PFAS Removal

| | | |
|---|---|---|
| | Pre-filtration | Post-filtration |
| Analyte | Concentration | Concentration |

Breast milk

| | | |
|---|---|---|
| PFOA | 4352 ppt | Non-Detectable |

Water³⁶

| | | |
|---|---|---|
| PFOA | 10⁶ ppt | >90% |
| PFOS | 10⁶ ppt | >90% |
| GenX | 10⁶ ppt | >90% |

TABLE 3
Preliminary Results - Nutritional Content
of Breast milk Pre- and Post-filtration

| | | |
|---|---|---|
| | Pre-filtration | Post-filtration |
| Analyte | Concentration | Removal |

| | | | | |
|---|---|---|---|---|
| Calories | 62 | kcal/dl | 60 | kcal/dl |
| Carbohydrates | 8.9 | g/dl | 8.7 | g/dl |
| Fat | 2.3 | g/dl | 2.1 | g/dl |
| Protein | 1.1 | g/dl | 0.9 | g/dl |

The filtration device may be able to reduce PFAS concentration in breast milk to protective levels, as preliminary data shows that the filter media reduces PFAS concentrations in breast milk to below the limit of detection and may remove more than 90% of PFAS compounds in water at concentrations comparable to that found in breast milk.

Furthermore, the filter will not negatively (or minimally) impact the nutritional quality of breast milk, as the preliminary data indicates that changes in calories, carbohydrate, fats, and proteins pre- and post-filtration are not significant, as Table 3 demonstrates.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A filtration assembly configured to filter PFAS from breast milk, comprising:
a filtration device configured to be positioned between a first collection unit and a second collection unit, wherein the filtration device is coupled to at least one of the first collection unit or the second collection unit and either including a quantity of breast milk;
the quantity of breast milk possibly containing a quantity of PFAS, wherein the filtration device is integrated with a filter, selectively positioned within the filtration device, to reduce the quantity of PFAS contained within the breast milk, wherein the quantity of breast milk is transferred from one collection unit to another; and
the quantity of breast milk processes through the filter and reduces the quantity of PFAS contained within the breast milk.

2. The filtration assembly of claim 1, wherein the first collection unit is a breast pump, such that the filtration device is positioned in-line with the breast pump to deliver the quantity of breast milk through the filter while the breast pump is in operation; and
the second collection unit to receive the quantity of breast milk.

3. The filtration assembly of claim 1, wherein the first collection unit is a first bottle and the second collection unit is a second bottle, such that the quantity of breast milk is transferred from one bottle to the other and passes through the filter.

4. The filtration assembly of claim 1, wherein the filtration device can define a first threading at a first end and a second threading at a second end, opposite the first end, configured to threadably couple a threaded portion of the first collection unit and the second collection unit, respectively, to form a liquid seal.

5. The filtration assembly of claim 1, wherein the filtration device relies on at least one of gravity or pressurization within the filtration device to process the quantity of breast milk through the filter.

6. The filtration assembly of claim 1, wherein the filter includes cyclodextrin.

7. The filtration assembly of claim 6, wherein the filter reduces the quantity of PFAS from the breast milk without significantly changing the nutritional profile of the breast milk.

8. The filtration assembly of claim 7, wherein the without significantly changing the nutritional profile of breast milk includes reducing the quantity of PFAS concentration by 5% or less.

9. The filtration assembly of claim 1, wherein a valve is integrated into the filtration device and is configured to release gas trapped within the first collection unit, second collection unit or the filtration device.

10. The filtration assembly of claim 1, wherein the filter is integrated with a brace configured to retain the filter in position relative to the filtration device during use.

11. The filtration assembly of claim 1, wherein the filter is a composed, compact disc and readily inserted and removed from the filtration device.

12. A method of filtering PFAS from breast milk, comprising:
positioning a filtration device between a first collection unit and a second collection unit, wherein the filtration device is coupled to at least one of the first collection unit or the second collection unit and either including a quantity of breast milk;
transferring the quantity of breast milk from one collection unit to another, the quantity of breast milk passing through a filter selectively positioned within the filtration device, wherein the filter reduces a quantity of PFAS possibly contained in the quantity of breast milk; and
reducing the quantity of PFAS from the quantity of breast milk.

13. The method of claim 12, further comprising positioning the filtration device in-line with a breast pump, the breast pump being the first collection unit;
delivering the quantity of breast milk from the breast pump through the filter; and
receiving the quantity of breast milk by a collection bottle, the collection bottle being the second collection unit.

14. The method of claim 12, further comprising threadably coupling the filtration device at a first end with the first collection unit; and threadably coupling the filtration device at a second end, opposite the first end, with the second collection unit, wherein the threadable connections form a liquid seal.

15. The method of claim 12, further comprising including cyclodextrin within the filter.

16. The method of claim 15, further comprising reducing the quantity of PFAS from the breast milk without significantly altering the nutritional profile of the breast milk.

17. The method of claim 16, further comprising only reducing the quantity of PFAS from the breast milk by 5% or less.

18. The method of claim 12, further comprising integrating a valve with the filtration device for automatic releasing of gas trapped within the first collection unit, the second collection unit or the filtration device.

19. The method of claim 12, further comprising integrating a brace with the filter for retaining the filter in position relative to the filtration device during use.

20. The method of claim 12, further comprising constructing the filter as a composed, compact disc for readable insertion and removal from the filtration device.
